(19) United States
(12) Patent Application Publication
(10) Pub. No.: US 2010/0191286 A1
(43) Pub. Date: Jul. 29, 2010

(54) FACET COMPRESSION SYSTEM AND RELATED SURGICAL METHODS

(76) Inventor: Jesse P. BUTLER, Morton Grove, IL (US)

Correspondence Address: Clements Bernard PLLC, 1901 Roxborough Road, Suite 250, Charlotte, NC, 28211, US

(21) Appl. No.: 12/572,661

(22) Filed: Oct. 2, 2009

Related U.S. Application Data
(63) Continuation of application No. 61/102,579, filed on Oct. 3, 2008.

(51) Int. Cl. A61B 17/70; A61B 17/88

(52) U.S. Cl. 606/247; 606/279

(57) ABSTRACT

The present invention provides a facet compression system, including: an articulating facet washer; one or more of a rigid, semi-rigid, and flexible cable coupled at its proximal end to the articulating facet washer; and one of a deployable and a deployed anchoring device coupled to a distal end of the cable; wherein the cable is selectively tensioned at the articulating facet washer, thereby compressing and immobilizing a facet joint of a spine of a patient. Optionally, the articulating facet washer is operable for pivoting in multiple planes relative to a structure disposed therethrough. Optionally, the articulating facet washer is operable for rotating relative to a structure disposed therethrough. Optionally, the deployable anchoring device includes an overwrapped compression cap. Optionally, the cable is selectively tensioned at the articulating facet washer via one or more of a zip tie ratchet mechanism, a crimping mechanism, and a screw mechanism.

### CROSS-REFERENCE TO RELATED APPLICATION(S)

The present non-provisional patent application/patent claims the benefit of priority of U.S. Provisional Patent Application No. 61/102,579, filed on Oct. 3, 2008, and entitled “FACET COMPRESSION SYSTEM AND RELATED SURGICAL METHODS,” the contents of which are incorporated in full by reference herein.

### FIELD OF THE INVENTION

The present invention relates generally to a minimally-invasive, surgically-implantable spinal system. More specifically, the present invention relates to a facet compression system that is used to immobilize a facet joint of the spine and related surgical methods.

### BACKGROUND OF THE INVENTION

In various cases, it is desirable to immobilize a facet joint of the spine (e.g. in support of a spinal fusion, etc.) by means other than conventional systems and methods. A variety of minimally-invasive, surgically-implantable systems have been developed in order to address such cases. Typically, these systems utilize a single transfacet bolt or the like that is disposed through a bore drilled through the superior and inferior facets of the facet joint. Alternatively, these systems utilize a facet cap, band, or the like that is disposed about the superior and inferior facets of the facet joint. However, not all spinal morphologies support the utilization of such a transfacet bolt, cap, or band, and the utilization of such a transfacet bolt, cap, or band can be clumsy. In such cases, the use of an alternative system is desirable. However, no such alternative systems are currently available or in existence.

### BRIEF SUMMARY OF THE INVENTION

In various exemplary embodiments, the present invention provides a facet compression system that is used to immobilize a facet joint of the spine and related surgical methods. In general, this facet compression system incorporates a cable or rod that is threaded or placed through and across the facet joint (via a bore drilled therethrough), anchored at one end by an enlarged structure. An articulating washer is then “snugged” onto the other end to engage that facet, thereby tensioning the system and compressing an immobilizing the facet joint. All placement orders of these components are contemplated herein.

In one exemplary embodiment, the present invention provides a facet compression system, including: an articulating facet washer; one or more of a rigid, semi-rigid, and flexible cable coupled at its proximal end to the articulating facet washer; and one of a deployable and a deployed anchoring device coupled to a distal end of the cable; wherein the cable is selectively tensioned at the articulating facet washer, thereby compressing and immobilizing a facet joint of a spine of a patient. Optionally, the articulating facet washer is operable for pivoting in multiple planes relative to a structure disposed therethrough. Optionally, the articulating facet washer is operable for rotating relative to a structure disposed therethrough. Optionally, the deployable anchoring device includes an overwrapped compression cap. Optionally, the cable is selectively tensioned at the articulating facet washer via one or more of a zip tie ratchet mechanism, a crimping mechanism, and a screw mechanism.

In another exemplary embodiment, the present invention provides a surgical method for placing a facet compression system, including: providing an articulating facet washer; providing one or more of a rigid, semi-rigid, and flexible cable coupled at its proximal end to the articulating facet washer; and providing one of a deployable and a deployed anchoring device coupled to a distal end of the cable; wherein the cable is selectively tensioned at the articulating facet washer, thereby compressing and immobilizing a facet joint of a spine of a patient. Optionally, the articulating facet washer is operable for pivoting in multiple planes relative to a structure disposed therethrough. Optionally, the articulating facet washer is operable for rotating relative to a structure disposed therethrough. Optionally, the deployable anchoring device includes an overwrapped compression cap. Optionally, the cable is selectively tensioned at the articulating facet washer via one or more of a zip tie ratchet mechanism, a crimping mechanism, and a screw mechanism.

In a further exemplary embodiment, the present invention provides a facet compression system, including: an articulating facet washer; one or more of a rigid, semi-rigid, and flexible cable coupled at its proximal end to the articulating facet washer; and an articulating anchoring device coupled to a distal end of the cable; wherein the cable is selectively tensioned at the articulating facet washer, thereby compressing and immobilizing a facet joint of a spine of a patient. Optionally, the articulating facet washer is operable for pivoting in multiple planes relative to a structure disposed therethrough. Optionally, the articulating facet washer is operable for rotating relative to a structure disposed therethrough. Optionally, the deployable anchoring device includes an overwrapped compression cap. Optionally, the cable is selectively tensioned at the articulating facet washer via one or more of a zip tie ratchet mechanism, a crimping mechanism, and a screw mechanism.

### BRIEF DESCRIPTION OF THE DRAWINGS

The present invention is illustrated and described herein with reference to the various drawings, in which like reference numbers are used to denote like assembly components/method steps, as appropriate, and in which:

FIG. 1 is a schematic diagram illustrating one step in a surgical method for placing a facet compression system of the present invention, namely the placement of an articulating facet washer adjacent to an inferior facet of a spine of a patient;

FIG. 2 is a schematic diagram illustrating another step in the surgical method for placing the facet compression system of the present invention, namely the withdrawal of a guide tube use to place the articulating facet washer;

FIG. 3 is a schematic diagram (two views) illustrating one exemplary embodiment of an articulating facet washer of the present invention; and

FIG. 4 is a schematic diagram illustrating a further step in the surgical method for placing the facet compression system of the present invention, namely the placement of a cable or rod through the articulating facet washer, the inferior facet, the associated facet joint, and the associated superior facet.

### DETAILED DESCRIPTION OF THE INVENTION

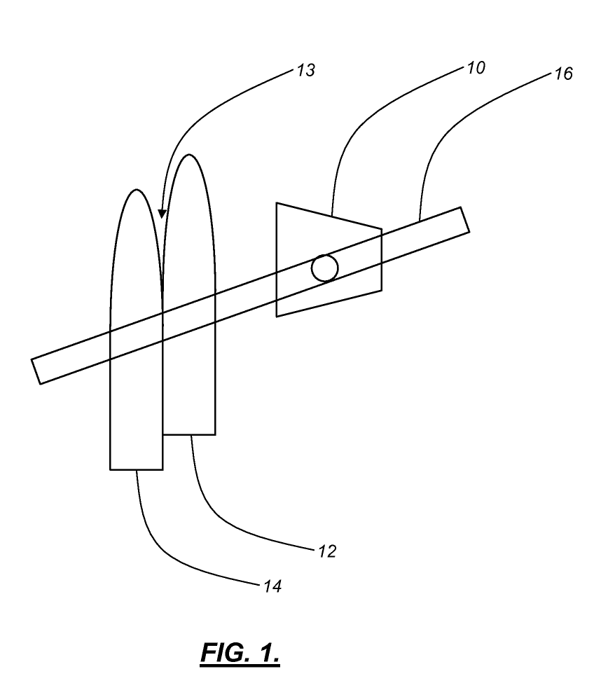

Referring now to FIG. 1, in one exemplary embodiment, the facet compression system of the present invention includes an articulating facet washer 10 that is selectively placed adjacent to an inferior facet 12 of a spine of a patient. The associated facet joint 13 of the spine of the patient includes the inferior facet 12, as well as a corresponding superior facet 14. The articulating facet washer 10 is placed by first drilling a bore or hole through the facet joint 13 at a predetermined angle that is convenient given the spinal morphology and access present. Optionally, this is done after distraction of the facet joint 13. A guide tube 16 is then placed through this bore or hole. The articulating facet washer 10 is engaged on this guide tube 16 and tapped into place. For example, the articulating facet washer 10 can be disposed concentrically about the guide tube 16 and translated along its length. As is described in greater detail herein below, the articulating facet washer 10 is configured and manufactured such that it can pivot in multiple planes relative to and/or rotate about the guide tube 16 or any other structure that it is engaged on. This is advantageous, as the preferred trajectory of placement is independent of where the articulating facet washer 10 is placed, and all spinal morphology and access issues can be addressed. In other words, the articulating facet washer 10 can always be made to “snugly” engage the inferior facet 12 adjacent to it.

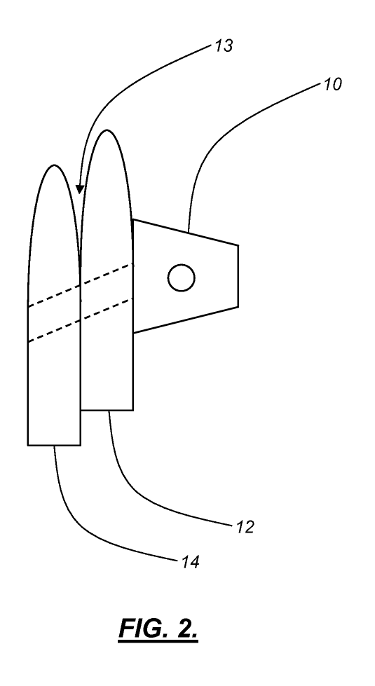

Referring now to FIG. 2, the articulating facet washer 10 is seated adjacent to the inferior facet 12 of the spine of the patient and the guide tube 16 has been withdrawn.

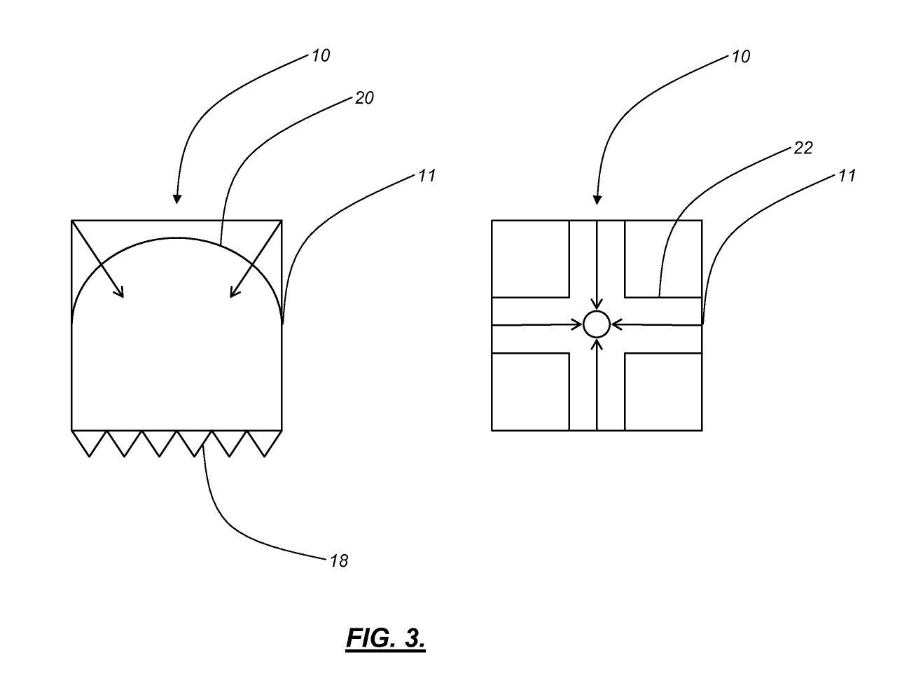

Referring now to FIG. 3, in one exemplary embodiment, the articulating facet washer 10 includes a body 11 and a facet engaging surface that has one or more cleats 18 or other raised structures, such that when the articulating facet washer 10 is seated, it will not easily move again. The body 11 can have any suitable shape, but is typically an annular structure. A ball 20 including a bore or a plurality of races 22 are disposed within/manufactured into the body 11 of the articulating facet washer 10, such that the guide tube 16 (FIG. 1) or any other structure that it is engaged on can pivot in multiple planes relative to the articulating facet washer 10. Other configurations and structures allowing this relative pivoting motion are also contemplated herein. Alternatively, the articulating facet washer 10 can be allowed to move relative to the inferior facet 12 (FIGS. 1 and 2) merely due to the inherent flexibility of the guide tube 16, in the case that a flexible guide tube 16 is utilized. The articulating facet washer 10 and guide tube 16 can also be correspondingly threaded, such that the articulating facet washer is “spun” along the guide tube 16.

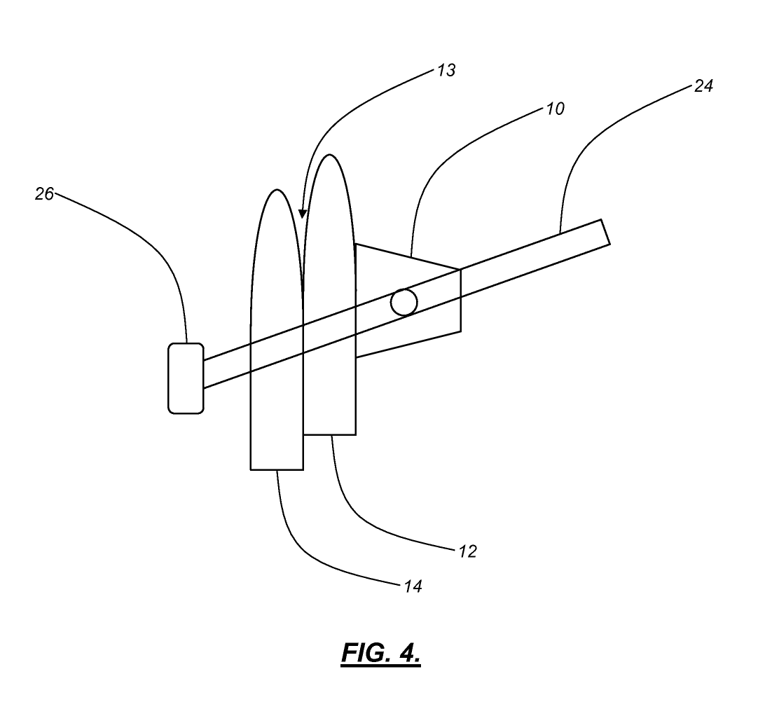

Referring now to FIG. 4, a cable 24 is placed through the articulating facet washer 10, the inferior facet 12, the facet joint 13, and the superior facet 14. This cable 24 can be placed with a delivery device and can be rigid (in the case of a rod), semi-rigid, flexible, consist of sections of different rigidity, etc. Once the cable 24 passes through the superior facet 14, an overwrapped compression cap 26 or the like is deployed, thereby anchoring the distal end of cable 24 through the facet joint 13. The proximal end of the cable 24 is then snugged and locked in the articulating facet washer 10 via an associated zip tie ratchet mechanism, crimping mechanism, screw mechanism, and/or the like, thereby anchoring, stabilizing, and immobilizing the facet joint 13 to any desired degree, with any desired compression torque being achieved. Finally, the feeds are cut. It should be noted that the cable/rod 24 and the guide tube 16 (FIG. 1) can be one and the same, serving both purposes and accomplishing both functions simultaneously.

In an alternative embodiment of the present invention, the placement site can be prepared, then a guide tube and integrated washer can be placed before or after drilling, then a guide wire can be placed and engage a distal washer, then the guide wire can be removed, and, finally, a transfacet bolt can be placed. The articulation features described above all apply with equal force to this alternative embodiment of the present invention.

Although the present invention has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the present invention, are contemplated thereby, and are intended to be covered by the following claims.

What is claimed is:

1. A facet compression system, comprising:
an articulating facet washer;
one or more of a rigid, semi-rigid, and flexible cable coupled at its proximal end to the articulating facet washer; and
one of a deployable and a deployed anchoring device coupled to a distal end of the cable;
wherein the cable is selectively tensioned at the articulating facet washer, thereby compressing and immobilizing a facet joint of a spine of a patient.

2. The facet compression system of claim 1, wherein the articulating facet washer is operable for pivoting in multiple planes relative to a structure disposed therethrough.

3. The facet compression system of claim 1, wherein the articulating facet washer is operable for rotating relative to a structure disposed therethrough.

4. The facet compression system of claim 1, wherein the deployable anchoring device comprises an overwrapped compression cap.

5. The facet compression system of claim 1, wherein the cable is selectively tensioned at the articulating facet washer via one or more of a zip tie ratchet mechanism, a crimping mechanism, and a screw mechanism.

6. A surgical method for placing a facet compression system, comprising:
providing an articulating facet washer;
providing one or more of a rigid, semi-rigid, and flexible cable coupled at its proximal end to the articulating facet washer; and
providing one of a deployable and a deployed anchoring device coupled to a distal end of the cable;
wherein the cable is selectively tensioned at the articulating facet washer, thereby compressing and immobilizing a facet joint of a spine of a patient.

7. The surgical method for placing the facet compression system of claim 6, wherein the articulating facet washer is operable for pivoting in multiple planes relative to a structure disposed there through.

8. The surgical method for placing the facet compression system of claim 6, wherein the articulating facet washer is operable for rotating relative to a structure disposed therethrough.

9. The surgical method for placing the facet compression system of claim 6, wherein the deployable anchoring device comprises an overwrapped compression cap.

10. The surgical method for placing the facet compression system of claim 6, wherein the cable is selectively tensioned at the articulating facet washer via one or more of a zip tie ratchet mechanism, a crimping mechanism, and a screw mechanism.

11. A facet compression system, comprising:
an articulating facet washer;
one or more of a rigid, semi-rigid, and flexible cable coupled at its proximal end to the articulating facet washer; and
an articulating anchoring device coupled to a distal end of the cable;
wherein the cable is selectively tensioned at the articulating facet washer, thereby compressing and immobilizing a facet joint of a spine of a patient.

12. The facet compression system of claim 11, wherein the articulating facet washer is operable for pivoting in multiple planes relative to a structure disposed therethrough.

13. The facet compression system of claim 11, wherein the articulating facet washer is operable for rotating relative to a structure disposed therethrough.

14. The facet compression system of claim 11, wherein the deployable anchoring device comprises an overwrapped compression cap.

15. The facet compression system of claim 11, wherein the cable is selectively tensioned at the articulating facet washer via one or more of a zip tie ratchet mechanism, a crimping mechanism, and a screw mechanism.
